(19) United States
(12) Patent Application Publication
(10) Pub. No.: US 2007/0025980 A1
(43) Pub. Date: Feb. 1, 2007

(54) Composition permitting muscle growth while protecting joint health

(76) Inventors: David L. Krogsgaard, Oceanside, CA (US); David A. Jenkins, Carlsbad, CA (US)

Correspondence Address: DUCKOR SPRADLING METZGER, 401 WEST A STREET, SUITE 2400, SAN DIEGO, CA, 92101-7915, US

(21) Appl. No.: 11/191,708

(22) Filed: Jul. 27, 2005

(51) Int. Cl. A61K 39/395; A61K 35/20

(52) U.S. Cl. 424/130.1; 424/535

(57) ABSTRACT

A composition which helps reduce joint inflammation and structural damage to joints during the growth of muscle structure, whether such growth is for repair of damaged muscle tissue or for development of new and enhanced muscular structure. The composition comprises a combination of edible nutritional protein, and a hyperimmune milk protein concentrate. The preferred nutritional protein is a whey protein isolate. The concentration of nutritional protein present in the composition typically ranges from about 10% by weight to about 90% by weight, more typically about 70% by weight to about 90% by weight. The composition may contain additional ingredients such as polypeptides, amino acids, antioxidants and various flavorings and sweeteners. A method of use of the composition is also disclosed.

### BACKGROUND OF THE INVENTION

1. Field of the Invention

The present invention pertains to composition which permits muscle growth while protecting joints, to recipes which include the composition, and to a method of using the composition.

2. Brief Description of the Background Art

There are a number of reasons for building muscle, ranging from a desire to increase strength and improve athletic capability, to achieving a desired appearance. The muscle growth may be in humans, or may be in other mammals where improved muscle structure will lead to improved task performance.

Muscle is attached at each end to a joint, to enable control and function of the joint. Muscle growth, and especially rapid muscle growth, frequently causes inflammation of the joints to which the muscle is attached. Depending on the amount of inflammation which occurs, the joints can become stressed to a point of long term injury. Further, there is a synergistic effect, since, as a joint is injured, the muscle growth slows, a natural reaction of the body trying to protect the joint.

Athletic trainers have noted that muscle growth problems, of the kind that cause muscle to be smaller and weaker on one side of the body than on the other, are frequently the result of an old joint trauma, or perhaps a recent joint injury. The team orthopedist for a major professional football team discovered that muscle wasting and deconditioning begins very early, within 24 hours after even a minor joint injury. Indications have been that muscle growth will not resume for months, if ever, if the related joint problems are not addressed.

The Center of Biomedical Engineering Research at the University of Delaware has found that when there is a joint problem, muscle function, even after recovery efforts was limited, with approximately 8% to 10% permanent activity loss. Surprisingly, the corresponding muscle on the other side of the body suffered a similar functional reduction to that on the side of the body which had experienced the joint problem. This may be another effort on the part of the body to balance stresses on structural mechanics within the body, as a means of preventing injury.

There has been a need for a method of maintaining joint health during muscle development, whether during repair of muscle function, or during growth and improvement of existing muscular structure.

### SUMMARY OF THE INVENTION

We have discovered a composition which helps reduce joint inflammation and structural damage to joints during the growth of muscle structure, whether such growth is for repair of damaged muscle tissue or for development of new and enhanced muscular structure. The composition comprises a combination of edible nutritional protein, and a hyperimmune milk protein concentrate. The preferred nutritional protein is a whey protein isolate. The concentration of nutritional protein present in the composition typically ranges from about 10% by weight to about 90% by weight, more typically about 70% by weight to about 90% by weight. A method of manufacturing a hyperimmune milk protein concentrate is described in detail in U.S. Pat. No. 5,650,175, which is hereby incorporated by reference in its entirety. The concentration of hyperimmune milk protein concentrate in the composition typically ranges from about 1% by weight to about 20% by weight, more typically about 7% by weight.

Polypeptides and amino acids of the kind which are related to muscle and nerve tissue development may be added optionally, for particular end use applications. Examples of polypeptides which may be added to the composition include glutamine peptides and hydrolized whey protein peptides having a molecular weight of less than about 10,000 daltons (typically having a molecular weight ranging from about 1,000 to about 10,000 daltons). Amino acids and their analogs and derivatives which may be added to the composition include phosphatidylcholine, L-Leucine, Isoleucine, Taurine, L-Glutamine, L-Arginine, and N-acetylcysteine, by way of example and not by way of limitation.

Antioxidants which provide antioxidant potentiating properties in the body are also typically, but optionally, included in the composition. Examples of such antioxidants include lecithin, lactoperoxidase, and lactoferrin, not by way of limitation.

A preservative of the kind known in the food industry may be added to provide freshness. A preferred preservative is tocopherol (vitamin e).

Various flavorings and sweeteners (preferably artificial sweeteners to reduce the amount of carbohydrates in the compound) may be added to provide palatability.

An exemplary composition may comprise whey protein isolate, hyperimmune milk protein concentrate, phosphatidylcholine, L-Leucine, Taurine, L-Glutamine, L-Argentine, hydrolyzed whey protein peptide having a molecular weight of less than 10,000 daltons (typically less than 2,000 daltons), Glutamine peptide, N-acetyl cesteine, Lactoperoxidase, and Lactoferrin. Flavorings such as vanilla, salt, and artificial sweetener may be added.

One skilled in the art of food and dietary supplements will recognize that there are a number of different flavorings and antioxidants which may be used in composition with the whey protein isolate, and hyperimmune milk protein concentrate. In addition, there are a variety of polypeptides and amino acids which are known to be beneficial to the body and which may be added to the composition as well, for particular remedial health benefits. It is not intended that the scope of the invention be limited to the exemplary composition described above.

The composition is typically provided in a serving size in the range of 30 grams. In one regimen of use, a consumer, upon initiating use of the compound, begins by using two servings per day, typically one in the morning and one at night for approximately 7 days. After this time period, the number of servings is reduced to one per day. Typically the single serving is taken in the morning, preferably after exercising.

### DETAILED DESCRIPTION OF EXEMPLARY EMBODIMENTS

As a preface to the detailed description presented below, it should be noted that, as used in this specification and the appended claims, the singular forms “a”, “an”, and “the” include plural referents, unless the context clearly dictates otherwise.

In instances where the word “about” is used in this document, this indicates that the precision of a value is within about ±10%.

We have developed a composition which permits muscle growth while protecting joints, have developed recipes which include the composition, and have developed a method of using the composition.

The composition comprises a combination of edible nutritional protein, and a hyperimmune milk protein concentrate. The preferred nutritional protein is a whey protein isolate. The concentration of nutritional protein present in the composition typically ranges from about 10% by weight to about 90% by weight, more typically about 70% by weight to 90% by weight. The whey protein may be present as two ingredients, where a first ingredient is a whey protein isolate in which the whey protein concentration is at least 90% by weight. The second ingredient may be a whey protein concentrate in which the whey protein concentration is about 80% by weight.

As discussed above, a method of manufacturing a hyperimmune milk protein concentrate is described in detail in U.S. Pat. No. 5,650,175. One example of a hyperimmune milk protein is available from Stolle Milk Biologics of Chicago, Ill., under the trade name MicroLactin™, by way of example and not by way of limitation. The concentration of hyperimmune milk protein concentrate in the composition typically ranges from about 1% by weight to about 20% by weight, more typically about 7% by weight.

Polypeptides and amino acids of the kind which are related to muscle and nerve tissue development may be added optionally, for particular end use applications. Examples of polypeptides which may be added to the composition include glutamine peptides and hydrolized whey protein peptides having a molecular weight of less than about 2,000 daltons (typically having a molecular weight ranging from about 1,000 to about 2,000 daltons), not by way of limitation. Amino acids and their analogs and derivatives which may be added to the composition include phosphatidylcholine, L-Leucine, Isoleucine, Taurine, L-Glutamine, L-Arginine, and N-acetylcysteine, by way of example and not by way of limitation. Typically the concentration of polypeptides in the composition ranges from about 0.01% by weight to about 5.0% by weight, more typically the concentration is in the range of about 0.02%. Typically the concentration of amino acids and their analogs and derivatives in the composition ranges from about 0.01% by weight to about 5.0% by weight, more typically the concentration is in the range of about 0.11% by weight to about 0.15% by weight.

Antioxidants which provide antioxidant potentiating properties in the body are also typically, but optionally, included in the composition. Examples of such antioxidants include lecithin, lactoperoxidase, and lactoferrin, not by way of limitation. Typically the concentration of antioxidants ranges from about 0.01% by weight to about 5.0% by weight in the composition, more typically the concentration is in the range of about 0.11% by weight to about 0.15% by weight.

A preservative of the kind known in the food industry may be added to provide freshness. A preferred preservative is tocopherol (vitamin e). The concentration of preservative typically ranges from about 0.01% by weight to about 1.0% by weight, more typically the concentration is in the range of about 0.01% by weight to about 0.015% by weight.

Various flavorings and sweeteners (preferably artificial sweeteners to reduce the amount of carbohydrates in the compound) may be added to provide palatability. For example vanilla flavorings are typically present at a concentration ranging from about 1% by weight to about 5% by weight. Artificial sweeteners are typically present at a concentration ranging from about 0.1% by weight to about 1.0% by weight, more typically about 0.11% by weight.

### EXAMPLES

### Example One

### Composition Ingredients and Method of Making the Composition

| | PERCENTAGE |
|---|---|
| INGREDIENTS | BY WEIGHT |
| The following ingredients are agglomerated * | |
| Whey Protein Isolate | 89.93 |
| Hyperimmune Milk Protein Concentrate | 7.0 |
| Whey Protein Concentrate (80% isolate) | 0.10 |
| The following are subsequently sprayed onto | |
| the agglomerated ingredients | |
| Phosphatidylcholine (surfactant, and choline | 1.0 |
| improves memory function) | |
| Coviox T-70 naturally occurring vitamin e, | 0.01 |
| available from Henkel Corp, Chicago, Ill. | |
| (antioxidant to preserve freshness) | |
| The following are post mixed into the | |
| agglomerated material. | |
| Natural and artificial vanilla (flavor) | 1.60 |
| Salt (flavor) | 0.10 |
| L-Leucine (increases protein synthesis, | 0.01 |
| decreases muscle sparing) | |
| Taurine (Cortisol reducing effects) | 0.01 |
| L-Glutamine (Increases protein synthesis, | 0.01 |
| decreases muscle sparing) | |
| L-Arginine (Anti Catabolic effects) | 0.10 |
| Acesulfame Potassium (artificial sweetener) | 0.06 |
| Sucralose (artificial sweetener) | 0.05 |
| Hydrolyzed whey protein peptide (rapidly | 0.01 |
| digested protein, rapid absorption, improves | |
| nitrogen retention) | |
| Glutamine peptides (rapidly digested protein, | 0.01 |
| rapid absorption, improves nitrogen retention) | |
| N-acetyl Cysteine (antioxidant) | 0.001 |
| Lactoperoxidase (antioxidant) | 0.001 |

* Agglomeration is carried out using a wetting and drying process in a fluid bed agglomerator of the kind commonly used in the industry.

The method described above for production of the composition provides a composition which is easily mixed into water using a spoon, where there is immediate dispersion and rapid dissolution of the compound.

The composition described above is particularly palatable.

The composition described above has been designated RX-98™- Vanilla for marketing purposes. Other products designated RX-98™ are available in multiple flavors.

### Example Two

### How to use the Composition

The composition may be used by the consumer in any manner which the consumer finds to be beneficial. However, I have determined that the following regimen of use is particularly beneficial. This a 4 phase regimen.

Phase 1: Body Cell Loading
For the first 7 days, take approximately 30 grams twice per day. Typically in the morning and in the evening.

Phase 2: Repair
For the next 14 days, take approximately 30 grams once per day.

Phase 3: Rebuild & Restore
For the next 28 days, take approximately 30 grams once per day.

Phase 4: Recovery
For lifelong results, take approximately 30 grams once per day for 6-7 days each week.

### Example Three

### How to Prepare the Composition

There are a number of ways to prepare the composition for consumption. It may be mixed with a spoon, shaker, or may be blended into a delicious shake with the consumer's favorite ingredients. For example, one serving (about 30 grams, which may be scooped from a container of the composition in granular form) may be added to a blender filled with 3-6 oz. of the consumer's favorite beverage. The blender cover is applied, and the mixture is blended for 20-30 seconds. For additional enjoyment, 1-2 ice cubes may be added, followed by additional blending for a time period of about 30 seconds. Another approach is to fill a shaker bottle with 3-6 oz. of the consumer's favorite beverage, add one serving of the composition, cover and shake for 20-30 seconds. An additional method of preparation is to add one serving to a tall glass, add 3-6 oz. of the consumer's favorite beverage, mix with a spoon for about 20 seconds or until the composition is well dispersed.

While the invention has been described in detail above with reference to several embodiments, various modifications within the scope and spirit of the invention will be apparent to those familiar with the industry of health care foods and supplements. Accordingly, the scope of the invention should be measured by the appended claims.

I claim:

1. A composition which is used to reduce joint inflammation and potential structural damage to joints during the growth of muscle structure, said composition comprising an edible nutritional protein and a hyperimmune milk protein concentrate.

2. A composition in accordance with claim 1, wherein said edible nutritional protein is a whey protein.

3. A composition in accordance with claim 2, wherein said whey protein is present in said composition at a concentration ranging from about 10% by weight to about 90% by weight.

4. A composition in accordance with claim 3, wherein said whey protein isolate is present at a concentration ranging from about 70% by weight to about 90% by weight.

5. A composition in accordance with claim 3, wherein said hyperimmune milk protein concentrate is present at a concentration ranging from about 1% by weight to about 20% by weight.

6. A composition in accordance with claim 5, wherein said hyperimmune milk protein concentrate is present at a concentration of about 7% by weight.

7. A composition in accordance with claim 4, wherein said hyperimmune milk protein concentrate is present at a concentration ranging from about 1% by weight to about 20% by weight.

8. A composition in accordance with claim 7, wherein said hyperimmune milk protein concentrate is present at a concentration of about 7% by weight.

9. A composition in accordance with claim 5, wherein polypeptides and amino acids of the kind which are beneficially related to muscle and nerve tissue development are present.

10. A composition in accordance with claim 7, wherein polypeptides and amino acids of the kind which are beneficially related to muscle and nerve tissue development are present.

11. A composition in accordance with claim 9, wherein antioxidants are present.

12. A composition in accordance with claim 10, wherein antioxidants are present.

13. A composition in accordance with claim 11, wherein flavorings are present.

14. A composition in accordance with claim 12, wherein flavorings are present.
